(19) United States
(12) Patent Application Publication
(10) Pub. No.: US 2005/0177006 A1
(43) Pub. Date: Aug. 11, 2005

(54) Catalytic process for preparing bisphenol A

(76) Inventors: Rainer Neumann, Tolz (DE); Ulrich Blaschke, Krefeld (DE); Stefan Westernacher, Kempen (DE)

Correspondence Address: BAYER MATERIAL SCIENCE LLC, 100 BAYER ROAD, PITTSBURGH, PA, 15205, US

(21) Appl. No.: 11/043,255

(22) Filed: Jan. 26, 2005

(30) Foreign Application Priority Data
Feb. 5, 2004 (DE) 1020040057222

(51) Int. Cl. C07C 39/16

(52) U.S. Cl. 568/728

(57) ABSTRACT

The present invention relates to a process for preparing bisphenol A in which the phenol and acetone are reacted to give bisphenol A in the presence of a sulfonic acid ion-exchanger in at least one reactor, and in which the reactor, at the time when it is started up, is filled with sulfonic acid ion-exchanger with a bulk volume of 30 to 70 vol. % of the total reactor volume.

### FIELD OF THE INVENTION

The present invention relates to bisphenol A and more particularly to a catalyzed process for its preparation.

### BACKGROUND OF THE INVENTION

Bisphenols, as condensation products of phenols and carbonyl compounds, are starting materials or intermediate products for preparing a number of commercial products. The condensation product from the reaction between phenol and acetone, 2,2-bis(4-hydroxyphenyl)propane (bisphenol A, BPA) is of particular industrial importance. BPA is used as the starting material for preparing a number of different types of polymeric materials such as, for example, polyarylates, polyetherimides, polysulfones and modified phenol/formaldehyde resins. Preferred areas of application are the preparation of epoxy resins and polycarbonates.

The preparation of bisphenols is well-known and is generally achieved by the acid catalyzed reaction of phenols with carbonyl compounds. The reaction is generally performed in fixed bed or fluidized bed reactors and also in reactive columns. In the case of BPA, reaction is achieved by reacting phenol with acetone, wherein a phenol/acetone ratio of greater than 5:1 is preferably used in the reaction. The reaction is performed in a continuous process, wherein the temperature is generally 45 to 110° C., preferably 50 to 80° C.

Sulfonated cross-linked polystyrene resins (acid ion-replacers or acid ion-exchangers, called ion-exchangers in the following) are preferably used as catalysts. Divinylbenzene is normally used as a cross-linker, but different cross-linkers, such as divinylbiphenyl, may also be used. Optionally, these ion-exchangers may be chemically modified by covalently or ionically bonded cocatalysts and are macroporous or gel-like, monodisperse or heterodisperse. However, the catalyst may also be an organic polymeric siloxane with sulfonic acid groups.

In the commercial form, acid ion-exchangers contain 45 to 85 wt. % of water. In order to avoid competition between water and the feedstocks at the catalytically active sites, and thus to avoid a reduction in activity of the ion-exchanger, the water, for example for use in the preparation of industrially important 2,2-bis(4-hydroxy)propane (BPA), should be largely removed. Also, as a result of the method of preparation, commercial ion-exchangers contain a certain proportion of acid oligomers which form decomposition products due to radical reactions of the polymer matrix. These are washed out during continuous passage of the reaction solution and have a negative effect on the purity, thermal stability and color of the products produced therewith. Therefore, such ion-exchangers are conditioned before first being used to prepare bisphenols.

DE-A-199 56 229 and DE-A-100 27 908 describe the conditioning of ion-exchangers which are used for the preparation of bisphenol A. In this process sulfonic acid ion-exchangers based on polystyrene, for example, are first washed with water and then the water is removed by rinsing with phenol. The ion-exchanger shrinks during removal of the water. When the water content decreases below a certain value due to displacement with phenol—the so-called isoswelling point—no further shrinkage takes place, even upon further decrease in the water content .

In addition, the ion-exchangers may also be modified with a cocatalyst, before or after conditioning or before or after first being used in the reaction. Co-catalysts used for this purpose are usually thiols, which carry at least one SH function. The co-catalyst can be both homogeneously dissolved in the reaction solution and, in the case of the acid ion exchangers, fixed on the catalyst itself. Homogeneous co-catalysts are e.g. mercaptopropionic acid, hydrogen sulfide, alkyl sulfides, such as e.g. ethyl sulfide, and similar compounds. Fixed co-catalysts are aminoalkyl thiols and pyridylalkyl thiols which are ionically bonded to the catalyst, it being possible for the SH function to be protected and only released during or after fixing to the catalyst. The co-catalyst can also be covalently bonded to the catalyst as alkyl or aryl thiol.

### SUMMARY OF THE INVENTION

A process for preparing bisphenol A is disclosed. The process entails reacting in at least one reactor phenol with acetone in the presence of a sulfonic acid ion-exchanger, characterized in that the bulk volume of the ion exchange resin fills at the start of the reaction 30 to 70% of the total volume of the reactor.

### DETAILED DESCRIPTION OF THE INVENTION

It has now been found that, during the preparation of bisphenols, in particular BPA from phenol and acetone with the aid of such catalysts, swelling of the catalyst takes place. This swelling may be so great that in some circumstances the original volume in the water-moist state, i.e. the original volume of the acid ion-exchanger in its commercial form containing 45 to 85 wt. % of water, is achieved once again.

When preparing bisphenols with this type of acid ion-exchanger in a fixed bed or fluidized bed reactor or also in reactive columns, it is advantageous, for economic reasons, that the available reactor volume be as full as possible with catalyst during preparation of the bisphenol.

However, it was observed that, during the preparation of bisphenols with such acid ion-exchangers in a fixed bed or fluidized bed reactor and also in reactive columns, that swelling of the acid ion-exchanger occurred. The reasons for this swelling process are not fully understood. On the one hand, it takes place due to inclusion of the water of reaction being produced, for example, in the reaction between acetone and phenol to yield BPA. It was found that the water content of the catalyst varies along the length of the reactor. In addition, apart from the desired product BPA, internal condensates of acetone and higher molecular weight compounds are also formed and these may be included in the catalyst and block the active sites. This process is also known as fouling or deactivation. This fouling process also apparently leads to swelling. As a result of recycling the stream of substances being produced when separating bisphenol from the reaction solution and comprising bisphenol A and so-called isomers (compounds known to a the person skilled in the art as being produced during the preparation of bisphenol A by condensation of phenol with acetone such as, for example, 2-(4-hydroxyphenyl)-2-(2-hydroxyphenyl)propane (o,p-bisphenol A), substituted indanes, hydroxyphenyl-indanols, hydroxyphenyl-chromanes, spirobisindanes, substituted indenols, substituted xanthenes and higher condensed compounds with three or more phenyl rings in the molecular structure, and others), water, cocatalyst and other impurities (for example internal condensates of acetone and reaction products of the impurities in the raw materials such as anisol, mesityl oxide, mesitylene and diacetone alcohol), this effect is evidently amplified even more. Further swelling is brought about by decrosslinking of the catalyst, for example by radical and/or iron salt-catalyzed processes.

Among other things, the activity, that is the ability of the carbonyl component to react with the phenol component to give bisphenol, decreases as a result of fouling. The reactivity of the ion-exchanger therefore decreases so that after a certain length of time the ion-exchanger has to be removed and replaced by fresh ion-exchanger.

It was observed that if the reactor is packed too full then, due to swelling, the ion-exchanger may enter the feed pipes and discharge pipes of the reactor and this may lead, for example, to a reduction in safety when operating the plant, due to blockage of the valves, and also to adverse effects on product quality if the ion-exchanger reaches the subsequent working-up steps for isolating the bisphenol.

It was found that it is advantageous to have filled the reactor, at the time when it is started up, with sulfonic acid ion-exchanger with a bulk volume of 30 to 70 vol. %, preferably 40 to 70 vol. %, particularly preferably 50 to 60 vol. %, of the total reactor volume.

The invention provides a process for preparing bisphenol A in which phenol and acetone are reacted to give bisphenol A in the presence of a sulfonic acid ion-exchanger in at least one reactor, and in which the reactor, at the time when it is started up, is filled with sulfonic acid ion-exchanger with a bulk volume of 30 to 70 vol. % of the total reactor volume.

The time when the reactor is started up means the time at which phenol and acetone are supplied to the reactor.

This level of filling avoids frequent and expensive stoppages in production in order to change, or remove by suction, catalyst which has swollen up over time.

In addition it has been found that it is also advantageous to measure the depth of packing of the sulfonic acid ion-exchanger in the reactor at suitable time intervals (e.g. regularly every 3 months) and optionally to withdraw some of the catalyst, in order to avoid the problems mentioned above.

In that case, it was observed that the removal of some of the upper portions of the catalyst from the reactor was advantageous, because the swelling had progressed furthest there and the efficiency of the catalyst in these locations had decreased to the greatest extent, as compared to the activity of the catalyst at start-up. Replacement of the entire amount of catalyst may thus be delayed.

According to a preferred embodiment of the inventive process, the top level of the sulfonic acid ion-exchanger in the reactor is checked at regular intervals and, on exceeding a preset maximum level in the reactor, some of the sulfonic acid ion-exchanger is removed so that the top level of the sulfonic acid ion-exchanger after removal is below the preset maximum value.

In another preferred embodiment, after removal of at least some of the ion-exchanger at the uppermost level in the reactor to reach an ion-exchanger volume below the preset maximum level the reactor is charged with fresh sulfonic acid ion-exchanger at maximum up to the preset maximum level.

After removing some of the swollen catalyst, the space which has been emptied may be filled entirely or partly with fresh catalyst which has already been conditioned. This has the additional advantage that more active catalyst is available for reaction.

Conditioning of the catalyst may take place either inside the actual reaction reactor or in a separate container.

### EXAMPLES

### Example 1 (According to the Invention)

A reaction solution comprising 4 wt. % acetone, 6 wt. % so-called isomers, 7 wt. % bisphenol A, 0.05 wt. % water, 300 ppm mercaptopropionic acid and 83 wt. % phenol is passed downwards through a reactor (height of reactor: 800 cm; depth of the catalyst bed: 400 cm; degree of filling of reactor when started up: 50 vol. %) filled with 100 m³ of phenol-moist acid ion-exchanger Lewatit S C 104® from Bayer Chemicals AG at a throughput of 30 t/h. The depth of the packing in the reactor is measured at regular intervals.

At the time when it is determined by measurement that less than only 70 cm of empty space is still present in the reactor, due to swelling of the ion-exchanger, enough acid ion-exchanger is abstracted from above under suction for 100 cm of empty space to be present again. The ion-exchanger removed under suction is disposed of in an appropriate manner.

### Example 2 (According to the Invention)

Example 2 is performed in the same way as example 1, but with the difference that an empty space of 150 cm is created by abstraction under suction and then the reactor is topped up with fresh catalyst which has already been conditioned to leave an empty space of 100 cm.

Although the invention has been described in detail in the foregoing for the purpose of illustration, it is to be understood that such detail is solely for that purpose and that variations may be made therein by those skilled in the art without departing from the spirit and scope of the invention except as it may be limited by the claims.

1. A process for preparing bisphenol A comprising reacting in at least one reactor phenol with acetone in the presence of a sulfonic acid ion-exchanger, characterized in that the bulk volume of the ion exchange resin fills at the start of the reaction 30 to 70% of the total volume of the reactor.

2. The process of claim 1 wherein the bulk volume of the ion exchange resin fills 40 to 70% of the total volume of the reactor.

3. The process of claim 1 wherein the bulk volume of the ion exchange resin fills 50 to 60% of the total volume of the reactor.
